(19) United States
(12) Patent Application Publication
(10) Pub. No.: US 2009/0104130 A1
(43) Pub. Date: Apr. 23, 2009

(54) COMPOSITIONS COMPRISING ORGANIC AND INORGANIC PHOSPHORS FOR CONVERTING ELECTROMAGNETIC RADIATION AND METHODS FOR USING THE SAME

(76) Inventors: Eric F. Bernstein, Gladwyne, PA (US); Howard Y. Bell, Princeton, NJ (US); Valery Belov, Allentown, NJ (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/104,047

(22) Filed: Apr. 16, 2008

Related U.S. Application Data
(60) Provisional application No. 60/911,962, filed on Apr. 16, 2007.

(51) Int. Cl. A61K 8/19; A61Q 17/00

(52) U.S. Cl. 424/59

(57) ABSTRACT

There is disclosed a topical composition comprising at least one organic and/or inorganic phosphor in a physiologically acceptable medium, wherein the phosphor(s) is present in an amount effective to convert electromagnetic radiation of an initial frequency to a different frequency. In one embodiment, the phosphors convert the initial radiation frequency, such as infrared or visible light, to a higher frequency, such as ultraviolet (UV) radiation. In another embodiment, the phosphors convert the initial radiation from a higher frequency, such as UV radiation, to a lower frequency, such as infrared or visible light. Methods of treating keratinous material, such as the skin, hair, or lips, using the disclosed compositions are also disclosed.

This application claims the benefit of domestic priority to U.S. Provisional Application 60/911,962, filed Apr. 16, 2007, which is herein incorporated by reference in its entirety.

The present invention relates to a topical composition comprising organic or inorganic phosphors that convert an incident radiation to a different radiation, such as up-converting electromagnetic radiation of a frequency (A) to a higher frequency (B) or vice versa. The present invention also relates to methods for using these compositions.

Topical compositions, such as cosmetic compositions, routinely comprise discrete elements to impart various properties to the composition, the surface to which they applied, or both. It is known that properties such as color, texture, viscosity and durability can be altered by adding discrete metallic, ceramic or organic elements to cosmetics.

Cosmetic or dermatological compositions for sunscreens are particularly reliant on the use of discrete particles to achieve a certain effect. In particular, inorganic, semi-metallic, and/or metallic oxide particles, such as ZnO and/or TiO₂ particles, are routinely added to block harmful radiation of sun from reaching the skin.

While the prior art, not to mention store shelves, are filled with topical compositions, such as formulations for skin care, hair care, sun care, sunscreens, oral care, color cosmetics, and personal cleaning, that have discrete particles for all kinds of benefits, none describe the use of discrete elements that converts electromagnetic energy of one energy to an innocuous, or even therapeutic energy for the underlying keratinous material.

With that in mind, the inventors have sought to develop a topical composition that converts the electromagnetic energy that impinges almost constantly on exposed keratinous material to a more beneficial energy. To that end, they have investigated the use of organic or inorganic phosphor in a topical composition.

A phosphor is a substance that exhibits the phenomenon of phosphorescence, or a sustained glowing after exposure to light or energized particles such as electrons. Phosphors have a finite emission time, with persistence being inversely proportional to wavelength. Because the persistence of the phosphor increases as the wavelength decreases, it is known that red and orange phosphors do not have sufficiently long glow times.

The organic and inorganic phosphors used in the present invention differ from these traditional phosphors in that they have an indefinite glow time. In addition, they have the ability to transfer electromagnetic energy of one frequency to a higher frequency (referred to as “up-converting”) or to a lower frequency (referred to as “down-converting”), depending on the rare earth metal used. A description of such phosphors is provided U.S. Pat. No. 5,698,397, which is herein incorporated by reference. This patent describes the use of such phosphors for biological and other assays.

Up-converting crystals, which take light or electromagnetic radiation of one frequency and convert it to light of a higher frequency (thus shorter wavelength), appear to contradict a basic law of physics directed to conservation of energy. However, two, four or more photons of a lower frequency or longer wavelength are converted into a single photon of higher frequency or shorter wavelength. Thus a number of photons of lower energy combine to produce one photon of higher energy. These compounds can emit visible light when irradiated with infra-red light.

In contrast, down-converting crystals take light or electromagnetic radiation of one frequency and convert it to light of a lower frequency (thus longer wavelength). These compounds can emit red or IR light when irradiated with UV or visible light.

The Inventors have surprisingly discovered that when incorporated into a topical composition, such as a cosmetic or dermatological composition, up-converting or down-converting crystals can change the frequency of an undesirable electromagnetic radiation that is impinging on the exposed skin to a desirable frequency.

### SUMMARY OF INVENTION

Thus, in one embodiment, the present disclosure is directed to a topical composition comprising organic and/or inorganic phosphors that convert electromagnetic radiation of one frequency, to electromagnetic radiation of a different frequency.

The present disclosure is also directed to a topical composition for the skin and/or hair comprising, in a physiologically acceptable medium, up-converting organic and/or inorganic phosphors that convert electromagnetic radiation of a frequency (A), such as IR radiation, to a higher frequency (B), such as visible light.

The present disclosure is also directed to a topical composition for the skin and/or hair comprising, in a physiologically acceptable medium, down-converting organic and/or inorganic phosphors that convert electromagnetic radiation of a frequency (C), such as UV or visible radiation, to a lower frequency (D), such as red or IR radiation.

In yet another embodiment, the present disclosure provides a method for treating a keratinous material, such as the skin or hair, that comprises applying to the keratinous material, a topical composition comprising, in a physiologically acceptable medium, organic and/or inorganic phosphors that convert electromagnetic radiation of an initial frequency to a different frequency; and exposing or impinging onto the keratinous material the electromagnetic radiation having the initial frequency.

### BRIEF DESCRIPTION OF THE DRAWINGS

FIG. 1 is a schematic representation of a nanoparticle preparation setup.

FIG. 2 is a TEM image of as-prepared Y₂O₃:Yb,Er nanoparticles.

FIG. 3 is a histogram of size distribution of Y₂O₃:Yb,Er nanoparticles.

FIGS. 4a-c are XRD spectra of (a) as-prepared Y₂O₃:8% Yb, 6% Er nanoparticles; (b) 1000° C. annealed Y₂O₃:8% Yb, 6% Er nanoparticles; (c) commercial bulk Y₂O₃:Eu.

FIG. 5 shows photoluminescence spectra of Y₂O₃:8% Yb, 6% Er nanoparticles.

FIG. 6 is a TEM image of inorganic phosphors of the present invention coated with SiO₂.

FIG. 7 is a classic Dieke diagram showing spectra and energy levels of rare-earth ions in crystals.

### DETAILED DESCRIPTION OF THE INVENTION

### Definitions

Certain terms used herein are defined below:

“Topical composition” refers to a composition for administration to any accessible body surface, including any keratinous substance, such as the skin, hair, lips. Topical compositions are intended to include cosmetic, dermatological, therapeutic, personal care or any other composition applied to the body.

“Up-converting” refers to the ability to convert electromagnetic energy to a higher energy or shorter wavelength.

“Down-converting” refers to the ability to convert electromagnetic energy to a lower energy or longer wavelength.

“Physiologically acceptable medium” refers to a medium that is non-irritating and non-toxic to the body surface to which the topical composition is applied.

“At least one” as used herein means one or more and thus includes individual components as well as mixtures/combinations.

A “film,” as used herein, refers to a continuous coating, i.e., a coating without holes visible to the naked eye, which covers at least a portion of the substrate to which the composition was applied. Further, a film, as used herein, may have any thickness and is not restricted to a thin coating.

“Film-forming polymer” as used herein means a polymer which, by itself or in the presence of a film-forming auxiliary, is capable, after dissolution in at least one solvent, of forming a film on the substrate to which it is applied once the at least one solvent evaporates.

“Keratinous” used with “material,” or “substance” or “fiber” as defined herein may be human keratinous substances, and may be chosen from, for example, nails, facial skin (including the lips), body skin, and keratinous fibers such as eyelashes, eyebrows, and hair.

“Polymers” as defined herein comprise copolymers (including terpolymers) and homopolymers, including but not limited to, for example, block polymers, cross linked polymers, and graft polymers.

“Rheological agent” as used herein refers to a molecule or a composition which can change, i.e., increase or decrease, at least one property chosen from deformation and flow, in terms of stress, strain and/or time, of a composition to which the rheological agent is added.

“Substrate,” as used herein, includes, for example, a keratinous substance, as defined above, as well as any other surface to which a composition may be applied.

“Emulsion” as used herein, includes oil-in-water (o/w) or water-in-oil (w/o) type dispersion formulations intended for application to the skin, particularly lotions and creams providing cosmetic or therapeutic benefits. The emulsions may contain any of a number of desired “active” ingredients, including skin colorants, drug substances, such as anti-inflammatory agents, antibiotics, topical anesthetics, antimicrobics, keratolytics, skin protectants or conditioners, humectants, ultraviolet radiation absorbers, sunless tanning agents and the like, depending on the intended uses for the formulations.

It is to be understood that both the foregoing general description and the following detailed description are exemplary and explanatory only and are not restrictive of the invention, as claimed. Reference will now be made in detail to exemplary embodiments of the present invention.

Phosphors are usually made from a suitable host material, to which an activator is added. Suitable activators that may be used in the present invention include ytterbium, erbium, thulium, holmium, and combinations of these materials. Non-limiting examples of activator couples include ytterbium/erbium, ytterbium/thulium, and ytterbium/holmium.

Generally, host materials comprise oxides, halides, sulfides, and selenides of various rare earth metals. Suitable phosphor host materials that may be used in one embodiment of the present invention include gadolinium, yttrium, lanthanum, and combinations of these materials. Particular non-limiting embodiments of such crystal matrices which may comprise the host material include oxy-sulfides, oxy-fluorides, oxy-chlorides, or vanadates of various rare earth metals.

Non-limiting embodiments of the organic and/or inorganic phosphors that can be used as host materials in the present disclosure include sodium yttrium fluoride (NaYF₄), lanthanum fluoride (LaF₃), lanthanum oxysulfide (La₂O₂S), yttrium oxysulfide (Y₂O₂S), yttrium fluoride (YF₃), yttrium gallate, yttrium aluminum garnet (YAG), gadolinium fluoride (GdF₃), barium yttrium fluoride (BaYF₅, BaY₂F₈), gadolinium oxysulfide (Gd₂O₂S), calcium tungstate (CaWO₄), yttrium oxide:terbium (Yt₂O₃Tb), gadolinium oxysulphide: europium (Gd₂O₂S:Eu); lanthanam oxysulphide: europium (La₂O₂S:Eu); and gadolinium oxysulphide: promethium, cerium, fluorine (Gd₂O₂S:Pr,Ce,F).

Additional organic and/or inorganic phosphors that can be used as host materials in the present disclosure include Zn₂SiO₄:Mn; CaWO₄; (Zn,Cd)S:Cu; ZnS:Ag; ZnO:Zn; KMgF₃:Mn; (Zn,Cd)S:Ag; (Zn,Cd)S:Ag; ZnS:Ag; ZnS:Cu,Au,Al; YVO₄:Eu; Y₂O₂S:Eu; CaSiO₃:Mn,Pb; KMgF₃:Mn; ZnS:Cu; MgF₂:Mn; (Zn,Mg)F₂:Mn; Zn₂SiO₄:Mn,As; Zn₂SiO₄:Mn,In; Gd₂O₂S:Tb; La₂O₂S:Tb; Y₂O₂S:Tb; Y₂O₂S:Tb,Eu; Y₃Al₅O₁₂:Ce; Y₃(Al,Ga)₅O₁₂:Ce; Y₂SiO₅:Ce; Y₃(Al,Ga)₅O₁₂:Tb; ZnS:Ag; Y₂O₃:Eu; InBO₃:Eu; InBO₃:Tb; InBO₃:Eu+InBO₃:Tb+ZnS:Ag; InBO₃:Eu+InBO₃:Tb; Y₂O₂S:Yb; Y₂O₂S:Yb,Er; Y₂O₂S:Yb,Ho; La₂O₂S:Yb,Er; La₂O₂S:Yb; Gd₂O₂S:Yb,Ho; Gd₂O₂S:Yb,Er; Gd₂O₂S:Yb; Gd₂O₂S:Tb; Gd₂O₂S:Eu; Gd₂O₂S:Pr; La₂O₂S:Eu; La₂O₂S:Tb; Y₂O₂S:Tb; (Zn,Cd)S:Ag; CsI:Na; CsI:Tl; CaS:Eu,Sm; CaS:Ce,Sm, SrS:Eu,Sm; ZnS:Cu,Pb; ZnS:Cu,Pb,Mn.

Representative phosphors which may be used in the present composition, including some of those previously listed, along with their characteristic absorption colors (and wavelengths) are: Gd₂O₂S:Tb (P43), green (peak at 545 nm); Gd₂O₂S:Eu, red (627 nm); Gd₂O₂S:Pr, green (513 nm); Gd₂O₂S:Pr,Ce,F, green (513 nm); Y₂O₂S:Tb (P45), white (545 nm); Y₂O₂S:Tb red (627 nm); Y₂O₂S:Tb, white (513 nm); Zn(0.5)Cd(0.4)S:Ag green (560 nm); Zn(0.4)Cd(0.6)S:Ag (HSr), red (630 nm); CdWO₄, blue (475 nm); CaWO₄, blue (410 nm); MgWO₄, white (500 nm); Y₂SiO₅:Ce (P47), blue (400 nm); YAlO₃:Ce (YAP), blue (370 nm); Y₃Al₅O₂:Ce (YAG), green (550 nm); Y₃(Al,Ga)₅O₁₂:Ce (YGG), green (530 nm); CdS:In, green (525 nm); ZnO:Ga, blue (390 nm); ZnO:Zn (P15), blue (495 nm); (Zn, Cd)S:Cu,Al (P22G), green (565 nm); ZnS:Cu,Al,Au (P22G), green (540 nm); ZnCdS:Ag, Cu (P20), green (530 nm); ZnS:Ag (P11), blue (455 nm); Zn₂SiO₄:Mn (P1), green (530 nm); ZnS:Cu (GS), green (520 nm).

In one embodiment, the composition comprises phosphors, such as those disclosed herein, that convert IR radiation to desired visible light. For example, in one embodiment, the phosphors used are capable of changing IR radiation to red, orange, yellow, and/or green light since such light is known to be useful for skin rejuvenation-vein removal, pore reduction, wrinkle reduction, and acne treatment. In another embodiment, depending on the phosphor used, the resulting visible light can be blue, which has also been found to be a desirable light for treating acne.

As such, there is disclosed a topical composition comprising, in a physiologically acceptable medium, organic and/or inorganic phosphors capable of changing the frequency of electromagnetic radiation.

In one embodiment, the organic and/or inorganic phosphors are present in the disclosed composition in an amount effective to convert electromagnetic radiation of a frequency (A) to a higher frequency (B). While in theory, the up-converting crystals of this embodiment can convert any electromagnetic energy to a higher energy (or shorter wavelength), in one embodiment, the electromagnetic radiation of frequency (A) comprises infrared or visible light, and the frequency (B) comprises ultraviolet (UV) radiation chosen from UVA, UVB, and UVC.

In a different embodiment, the organic and/or inorganic phosphors are present in the disclosed composition in an amount effective to convert electromagnetic radiation of a frequency (C) to a lower frequency (D). While in theory, the down-converting crystals of this embodiment can convert any electromagnetic energy to a lower energy (or longer wavelength), in one embodiment, the electromagnetic radiation of frequency (C) comprises ultraviolet (UV) radiation, such as UVA, UVB, or UVC and the frequency (D) comprises infrared or visible light.

As evident, compositions comprising down-converting crystals can find particular utility is sunscreen compositions since they have the ability to convert potentially harmful UV radiation to innocuous or even beneficial radiation for the skin.

The organic and/or inorganic phosphors may be present in the cosmetic or dermatological composition in an amount ranging from 0.01% to 60% by weight, relative to the total weight of the composition, such as from 0.1% to 30% or even 1% to 15% by weight, relative to the total weight of the composition.

In one embodiment, the disclosed cosmetic composition may further comprise an activator for the organic and/or inorganic phosphors, such as a ytterbium containing activator. Non-limiting examples of the ytterbium containing activator include ytterbium/erbium, ytterbium/thulium, ytterbium/terbium, and ytterbium/holmium.

In another embodiment, the disclosed composition may further comprise one or more psoralen compounds or psoralen derivatives. Non-limiting embodiments of the psoralen compounds or psoralen derivatives include 8-methoxypsoralen (8-MOP), 5-methoxypsoralen (5-MOP), and tri-psoralen.

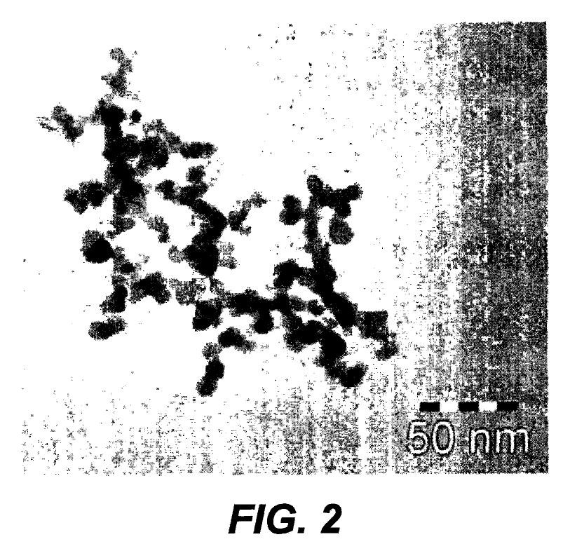

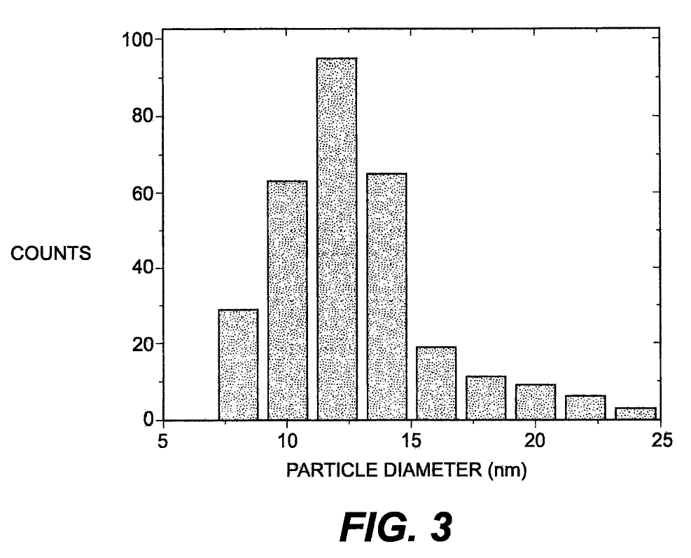

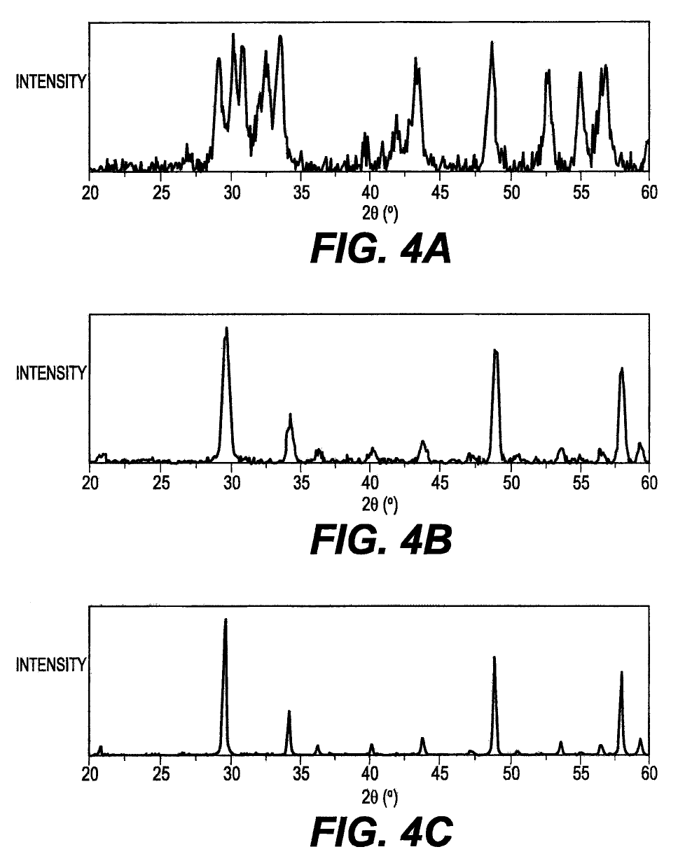

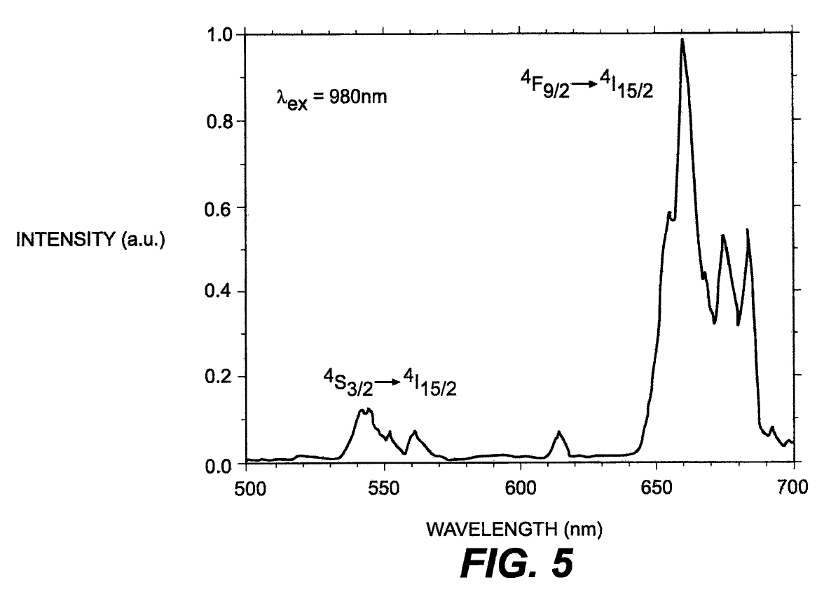

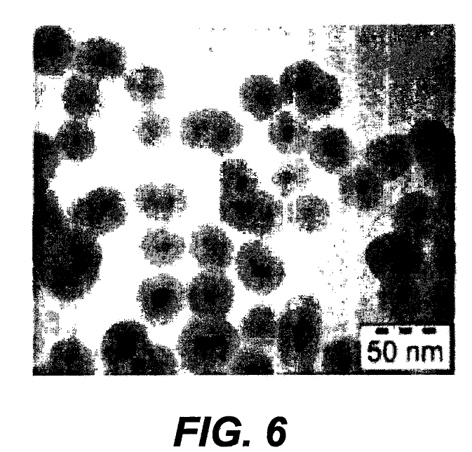

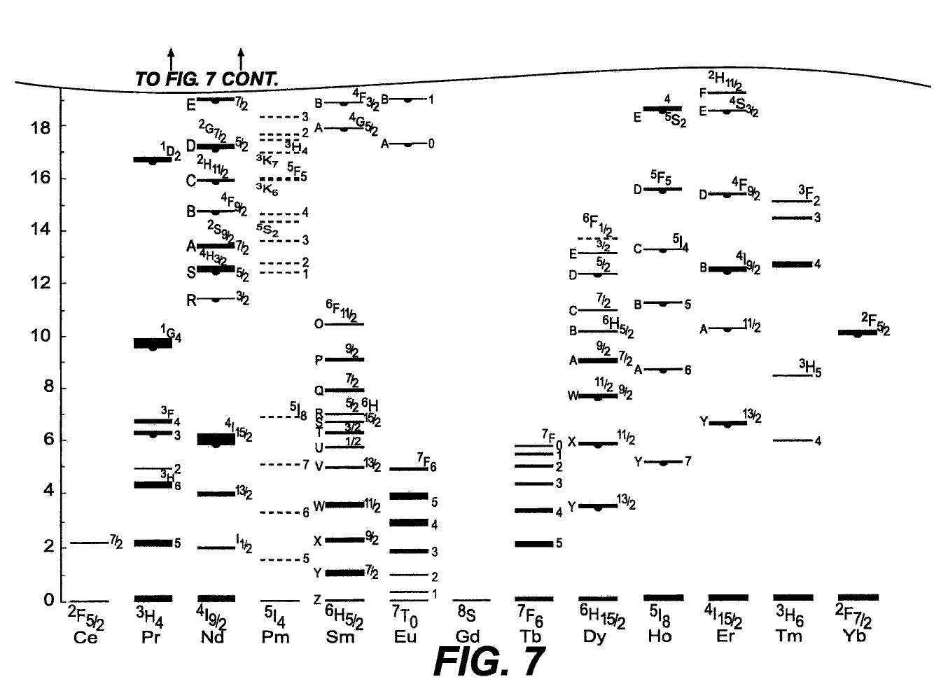

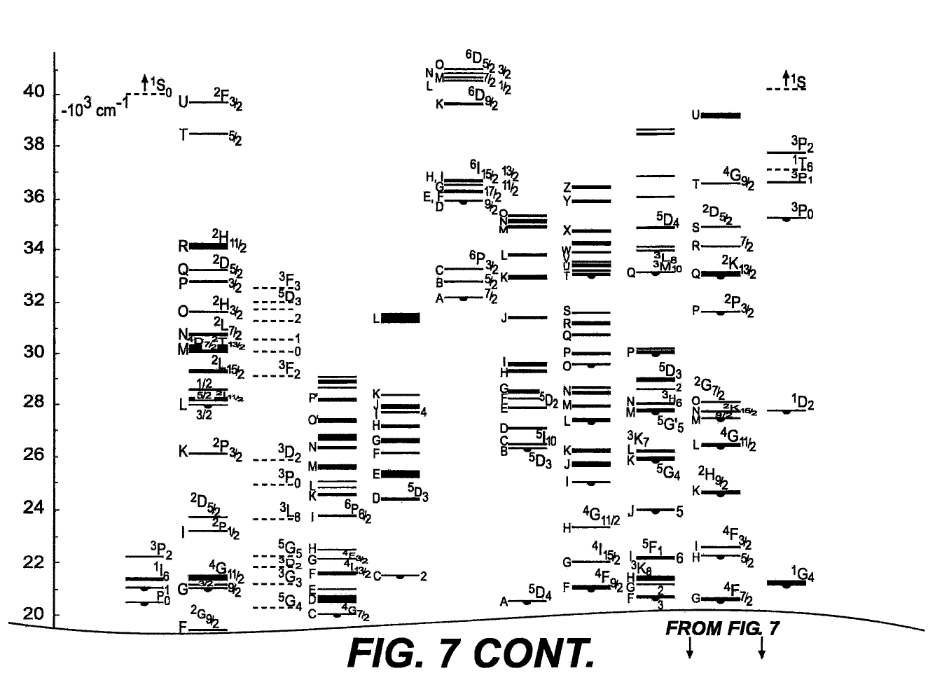

The organic and/or inorganic phosphors according to the present disclosure typically have an average particle size ranging from 1 to 1000 nanometers, such as from 5-100 nm, or even 10-50 nm. The concentration of the organic and/or inorganic phosphors in the inventive composition as well as in the above-defined regions and the size of the organic and/or inorganic phosphors can be measured by methods known for such which are well known in the art. For example, x-ray diffraction (XRD), scanning electron microscopy (SEM), transmission electron microscopy (TEM), and/or BET surface area analysis may be used. Examples of the particle sizes as measured from a TEM are shown in FIGS. 2 and 6.

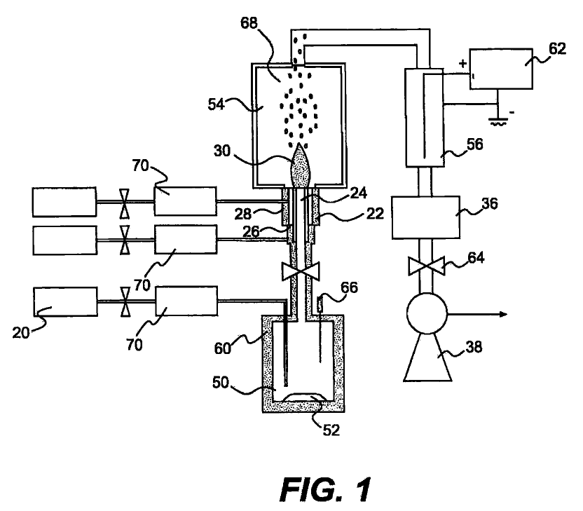

The organic and/or inorganic phosphors according to the present disclosure are typically synthesized from rare-earth doped phosphorescent oxide nanoparticles. The method further provides for homogeneous ion distribution through high temperature atomic diffusion, as described below with reference to FIG. 1.

FIG. 1 depicts a flame pyrolysis system comprising a vaporizing chamber 50 comprising a solid-phase precursor composition 52; a low pressure combustion chamber 54 that houses flame 30; and a particle collection subsystem comprising an electrostatic precipitator 56, a high voltage power supply 62, a cooling system 36, and a vacuum pump 38 for collecting synthesized nanoparticles.

A solid-phase precursor composition (hereinafter referred to as “the precursor composition”) is prepared by mixing one or more rare earth element dopant precursor powders with one or more oxide-forming host metal powders. Stoichiometric amounts of host metal and rare earth element are employed to provide rare earth element doping concentrations in the final particle of at least 0.5 mol % up to the quenching limit concentration.

In one embodiment, the quenching limit concentration is about 15-18 mol % for europium-doped Y₂O₃ nanoparticles, while it is about 10 mol % for erbium-doped Y₂O₃ nanoparticles. Also, for Yb and Er-codoped Y₂O₃ nanoparticles, the quenching limit depends upon the ratio of Yb:Er.

The rare earth element dopant precursor powders include, but are not limited to organometallic rare earth complexes having the structure:

RE(X)₃

wherein X is a trifunctional ligand and RE is a rare earth element. Any rare earth element or combinations thereof can be used (i.e., europium, cerium, terbium, dysprosium, holmium, erbium, thulium, ytterbium, lutetium) with particular mention being made to europium, cerium, terbium, holmium, erbium, thulium and ytterbium, as well as the following combinations: ytterbium and erbium, ytterbium and holmium and ytterbium and thulium.

Strontium can also be used, and for purposes of the present invention, rare earth elements are defined as including strontium. are earth element dopant precursor powders include Yb(TMHD)₃, Er(TMHD)₃, Ho(TMHD)₃, Tm(TMHD)₃, erbium isopropoxide (C₉H₂₁O₃Er), ytterbium isopropoxide (C₉H₂₁O₃Yb), and holmium isopropoxide (C₉H₂₁O₃Ho).

Examples of trifunctional ligands include tetramethylheptanedionate (TMHD), isopropoxide (IP), and the like.

The oxide forming host metal can be, but is not limited to, lanthanum, yttrium, lead, zinc, cadmium, and any of the Group II metals such as, beryllium, magnesium, calcium, strontium, barium, aluminum, radium and any mixtures thereof or a metalloid selected from silicon, germanium and II-IV semi-conductor compounds. Oxide-forming host metal powders include Y(TMHD)₃, Al(TMHD)₃, Zr(TMHD)₃, Y(IP), and Ti(IP).

The rare earth element dopant precursor powder and oxide-forming host metal powders are mixed in vaporizing chamber 50 to form the precursor composition 52. The vaporizing chamber 50 is heated to a temperature sufficient to vaporize the precursor composition 52. Once the precursor composition is vaporized, an inert carrier gas 20, such as, but not limited to, nitrogen, argon, helium, and mixtures thereof, transports the vaporized precursor composition 58 through a central tube 24 to a low pressure combustion chamber 54 that houses flame 30.

FIG. 1 depicts an embodiment wherein a co-flow burner 22 has three concentric tubes 24, 26, and 28. Central tube 24 transports vaporized precursor composition 58 to the low pressure combustion chamber 54, while tubes 26 and 28 co-deliver two reactive gases. In the depicted embodiment, tube 26 delivers methane and tube 28 delivers oxygen. The reactive gas inlets can be any size depending upon the desired gas delivery rate.

A flame produces active atomic oxygen via chain-initiation reaction of

H+O₂═OH+O (i)

A high concentration of oxygen in the flame activates and accelerates the oxidation of rare-earth ions and host materials through a series of reactions:

R+O→RO; (ii)

RO+O→ORO; and (iii)

ORO+RO→R₂O₃ (iv)

Reactions (ii) through (iv) are much faster than the oxidation reaction in low temperature processing represented by the reaction below;

2R+3/2O₂═R₂O₃ (v)

The reaction represented by formula (v) has a much higher energy barrier than the reactions in formulae (i)-(iv) in which radicals formed in flames diffuse and help produce faster ion incorporation.

Generally, in flame spray pyrolysis a higher flame temperature increases particle sintering and agglomeration. However, this was not the case in the current work as seen in FIG. 2 wherein spherical, discrete particles are seen. It is proposed that in addition to residence time, the initial size of the vapor-phase particles in the vaporized precursor composition and the precursor itself are the dominant factors that determine final particle size. As the vaporized precursor composition passes through the flame, it directly reacts and releases heat to the flame increasing flame temperature. Thus, a shorter flame residence time is needed, which allows for the production of smaller particles.

Temperatures between about 1800 and about 2900° C. are used in one embodiment, with temperatures between about 2200 and about 2400° C. being particularly noted. Temperatures within this range produce monodispersed rare earth doped activated oxide nanoparticles without significant agglomeration having an essentially uniform distribution of rare earth ions within the particles. Actual residence time will depend upon reactor configuration and volume, as well as the volume per unit time of vaporized precursor composition delivered at a given flame temperature. Cubic phase particles are obtained having an average particle size ranging from 5 to 50 nanometers, such as from 10 to 20 nanometers. Until recently, it was not possible to obtain activated cubic phase particles on a nanoscale. The particles also exhibit quenching limit concentrations heretofore unobtained.

The flame temperature can be manipulated by adjusting the flow rates of the gas(es). For example, the temperature of the flame can be increased by increasing the methane flow rate in a methane/oxygen gas mixture. Guided by the present specification, one of ordinary skill in the art will understand without undue experimentation how to adjust the respective flow rates of reactive gas(es) and inert carrier gas to achieve the flame temperature producing the residence time required to obtain an activated particle with a predetermined particle size.

Any reactive gas can be used singularly or in combination to generate the flame for reacting with the vaporized precursor composition, such as, but not limited to, hydrogen, methane, ethane, propane, ethylene, acetylene, propylene, butylenes, n-butane, iso-butane, n-butene, iso-butene, n-pentane, iso-pentane, propene, carbon monoxide, other hydrocarbon fuels, hydrogen sulfide, sulfur dioxide, ammonia, and the like, and mixtures thereof.

A hydrogen flame can produce high purity nano-phosphors without hydrocarbon and other material contamination. In the depicted embodiments, the flame length determines particle residence time within the flame. Higher temperatures produce satisfactory nanoparticles with shorter flames. Flame length is similarly manipulated by varying gas flow rates, which is also well understood by the ordinarily skilled artisan. Increasing the flame length increases the residence time of the particles in the flame allowing more time for the particles to grow. The particle residence time can be controlled by varying the different flow rates of the gases, and is readily understood by one of ordinary skill in the art guided by the present specification.

FIG. 1 shows a particle collection subsystem comprising an electrostatic precipitator 56, a high voltage power supply 62, a cooling system 36, and vacuum pump 38. The electrostatic precipitator 56 is connected to low pressure combustion chamber 54 for gathering the formed nano-phosphor particles 68. Vacuum pump 38 extracts gases and heat from the combustion chamber 54 through cooling system 36. Vacuum pump 38 also provides the force necessary to extract the formed nano-phosphor particles 68 from the combustion chamber 54 onto the electrostatic precipitator 56. A needle valve 64 installed between electrostatic precipitator 56 and vacuum pump 38 provides a means for controlling the pressure in low pressure combustion chamber 54.

The compositions according to the invention may further comprise at least one film-forming polymer. The at least one film-forming polymer may be dissolved or dispersed, for example, in the form of particles, in the cosmetically acceptable medium. Non-limiting examples of the at least one film-forming polymer include synthetic film-forming polymers including film-forming polymers formed via radical-mediated polymerization and film-forming polymers formed from polycondensation, and film-forming polymers of natural origin. In one embodiment, the compositions of the present invention do not include both an aqueous dispersion of at least one film forming polymer having a particle size ranging from 10 nm to 500 nm and at least one associative polyurethane.

Non-limiting examples of the at least one film-forming polymer include vinyl polymers (including vinyl copolymers), such as, for example, acrylic polymers. For example, vinyl film-forming polymers may be formed from polymerization of at least one monomer comprising at least one ethylenically unsaturated group and at least one additional group chosen from acid groups, ester groups, and amide groups.

One of ordinary skill in the art will recognize that the at least one film-forming polymer may be formed from any monomers known to those skilled in the art which fall within the categories of acrylic and vinyl monomers (including monomers modified with a silicone chain). In one embodiment, the at least one film-forming polymer is not chosen from a vinyl-silicone graft or block copolymer comprising a silicone polymer segment and a vinyl polymer segment which is prepared by the free radical polymerization of a mercapto functional silicone chain transfer agent and vinyl monomers.

Non-limiting examples of acrylic film-forming polymers in aqueous dispersion which can be used according to the present invention include those sold by Zeneca under the tradenames Neocryl XK-90®, Neocryl A-1070®, Neocryl A-1090®, Neocryl BT62®, Neocryl A-1079® and Neocryl A-523®, and those sold by Dow Chemical under the tradename Dow Latex 432®.

Suitable film-forming polymers formed via polycondensation which can be used as the at least one film-forming polymer may be anionic film-forming polymers, cationic film-forming polymers, nonionic film-forming polymers or amphoteric film-forming polymers.

Non-limiting examples of film-forming polyurethane polymers in aqueous dispersion include polyester-polyurethanes sold under the tradenames “Avalure UR-405®”, “Avalure UR-410®”, “Avalure UR425®”, “Avalure UR-450” and “Sancure 2060®” by Goodrich; polyether-polyurethanes sold under the tradename “Sancure 878®” by Goodrich; and polyether-polyurethanes sold under the tradename “Neorez R 970®” by Zeneca.

As previously mentioned, the at least one film-forming polymer may be chosen from film-forming polymers of natural origin. Film-forming polymers of natural origin, as used herein, may optionally be modified. Non-limiting examples of film-forming polymers of natural origin include cellulose polymers such as nitrocelluloses, cellulose acetates, cellulose acetobutyrates, cellulose acetopropionates, and ethylcelluloses.

According to the present invention, the at least one film-forming polymer, if present, may be present in an amount generally ranging, for example, from 1% to 50% by weight, relative to the total weight of the composition, such as from 5% to 40%. One of ordinary skill in the art will recognize that the at least one film-forming polymer according to the present invention may be commercially available, and may come from suppliers in the form of a dilute solution. The amounts of the at least one film-forming polymer disclosed herein therefore reflect the weight percent of active material.

In addition to or alternative to a physiologically acceptable medium, the disclosed composition may be dispersed in a cosmetically acceptable medium, which refers to a medium that may be applied to at least one keratinous substance. Either of these mediums may be chosen from at least one solvent, water and mixtures thereof. Non-limiting examples of the at least one solvent include organic solvents. Non-limiting examples of the acceptable medium include aqueous-alcoholic mixtures such as mixtures comprising at least one C₁-C₅ monoalcohol.

Non-limiting examples of organic solvents include ketones; alcohols; glycols; propylene glycol ethers; short-chain esters having from 3 to 8 carbon atoms; ethers; alkanes; cyclic aromatic compounds; and aldehydes.

According to the present invention, the physiologically or cosmetically-acceptable medium may be present in an amount generally ranging from 15% to 99% by weight, relative to the total weight of the composition, such as from 50% to 75%.

The compositions of the present invention may further comprise at least one film-forming auxiliary agent. The at least one film-forming auxiliary agent may improve at least one film-forming property of the composition, such as at least one film-forming property chosen from at least one film-forming property of the base composition and at least one film-forming property of the surface composition. When at least one film-forming auxiliary agent is used with at least one film-forming polymer, the at least one film-forming auxiliary agent can be chosen from any compound known to those skilled in the art as being capable of modifying, such as enhancing, at least one property of the at least one film forming polymer.

In one embodiment, the at least one film-forming auxiliary agent is chosen from plasticizers. Plasticizers are used in the art for the purposes of softening and plasticizing the film-formers in order to provide better flexibility. Non-limiting examples of known plasticizers include tricresyl phosphate, benzyl benzoate, tributyl phosphate, butyl acetylricinoleate, glyceryl acetylricinoleate, dibutyl phthalate, butyl glycolate, dioctyl phthalate, butyl stearate, tributoxyethyl phosphate, triphenyl phosphate, triethyl citrate, tributyl acetylcitrate, 2-triethylhexyl acetylcitrate, dibutyl tartrate, dimethoxyethyl phthalate, diisobutyl phthalate, diamyl phthalate, camphor, glycerol triacetate, and glycerol tribenzoate.

In another embodiment, the at least one film-forming auxiliary agent is chosen from coalescers. For example, when the at least one film-forming polymer is in the form of particles dispersed in the at least one cosmetically acceptable medium, the at least one film-forming auxiliary agent may be chosen from coalescers.

The at least one film-forming auxiliary agent, if present, may be present in an amount generally ranging, for example, from 0.1% to 15% by weight relative to the total weight of the composition, such as from 0.5% to 10%.

According to the present invention, the composition may further comprise at least one coloring agent. Non-limiting examples of the at least one coloring agent include lipophilic dyes (liposoluble dyes), hydrophilic dyes, pulverulent dyestuffs including traditional pigments (including interferential and non-interferential pigments), nacres, glitters and flakes usually used in cosmetic or dermatological compositions, and mixtures thereof. The at least one coloring agent, if present, may be present in the composition in an amount generally ranging from 0.01% to 50% by weight, relative to the total weight of the composition, such as from 0.01% to 30%.

The composition according to the present invention may further comprise at least one additive known to those skilled in the art as being capable of being incorporated into cosmetic compositions. Non-limiting examples of the at least one additive include vitamins, minerals, thickeners, fillers, spreading agents, thixotropes, rheological agents, wetting agents, dispersants, anti-foaming agents, preserving agents, UV screening agents, UV absorbing agents, active agents, surfactants, moisturizers, fragrances, neutralizing agents, stabilizers, proteins and antioxidants.

The compositions of the invention are formulated according to techniques that are well known to this art, advantageously for the preparation of emulsions of oil-in-water (o/w) type.

For example, for the disclosed compositions may comprise a support of oil-in-water emulsion type, the aqueous phase (in particular comprising the hydrophilic screening agents) generally constitutes from 50% to 95% by weight, such as from 70% to 90% by weight, relative to the total weight of the composition, and the oily phase (in particular comprising the lipophilic screening agents) generally constitutes from 5% to 50% by weight, such as from 10% to 30% by weight, relative to the total weight of the composition.

The compositions of this invention may also comprise conventional cosmetic additives and adjuvants selected, in particular, from among fatty substances, organic solvents, thickeners, softeners, opacifiers, stabilizers, colorants, emollients, antifoams, moisturizers, fragrances, preservatives, polymers, fillers, sequestering agents, bactericides and/or odor absorbers, acidifying or basifying agents, surfactants, free-radical scavengers, antioxidants, vitamins such as vitamins E and C, alpha-hydroxy acids or any other ingredient conventionally formulated into cosmetics.

Exemplary fatty substances include an oil or a wax or mixtures thereof and they can also comprise fatty acids, fatty alcohols and fatty acid esters. The oils are advantageously selected from among animal, plant, mineral and synthetic oils and, in particular, from among liquid petroleum jelly, liquid paraffin, volatile or non-volatile silicone oils, isoparaffins, polyolefins, fluoro oils and perfluoro oils. Similarly, the waxes are advantageously selected from among animal, fossil, plant, mineral and synthetic waxes that are per se known.

The inventive compositions may, for example, be prepared by a sequential addition of all the ingredients or through several steps including the grinding of discrete particles, such as pigments, when present, in the presence of the up-converting crystals. One of ordinary skill in the art will recognize satisfactory equipment and procedures.

In one embodiment, there is disclosed a method for electromagnetically treating a keratinous surface, the method comprising: applying to at least one keratinous surface, the previously disclosed topical composition that comprises, in an acceptable medium, organic and/or inorganic phosphors for changing the frequency of radiation incident on the keratinous surface.

For example, there is disclosed a method comprising applying to at least one keratinous surface, a composition comprising the disclosed up-converting crystals present in an amount effective to convert electromagnetic radiation of a frequency (A) to a higher frequency (B). This method further comprises exposing the keratinous surface to a source of electromagnetic radiation (A).

In another embodiment, there is disclosed a method comprising applying to at least one keratinous surface, a composition comprising the previously disclosed down-converting crystals present in an amount effective to convert electromagnetic radiation of a frequency (C) to a lower frequency (D). This method further comprises exposing the keratinous surface to a source of electromagnetic radiation (C).

These disclosed methods can be useful for treating known skin conditions. For example, the appearance of skin aged by light includes wrinkles and lines, Irregular pigmentation, the formation of brown spots, keratoses and even carcinomas or malignant melanomas. Skin aged prematurely by everyday UV exposure is, moreover, characterized by lower activity of the Langerhans cells and slight, chronic inflammation. In one embodiment, the disclosed invention is directed to compositions and methods of reducing these appearances by altering the radiation impinging the keratinous material to which it is applied.

As explained in more detail below, the present disclosure describes compositions that serve to prevent UV-A and/or UV-B radiation from impinging on the skin. While the disclosure is directed to the conversion of dangerous UV-A and/or UV-B radiation to less harmful visible or IR radiation, it does not exclude the use of other more traditional mechanism that can be used in combination with up-conversion or down-conversion. Such traditional mechanisms include reflection and scattering of the rays at the surface of pulverulent solids, and absorption on chemical substances.

### Skin Care Compositions

The harmful effect of the ultraviolet part of solar radiation on the skin is generally known. Depending on their particular wavelength, the rays have different effects on the skin. For example, UV radiation with a wavelength between 100 and 280 nm (UV-C) is absorbed by the ozone layer in the Earth's atmosphere and accordingly is not found in the solar spectrum. It is therefore of no physiological importance during sunbathing. It is noted, however, that the inventive compositions can be used to protect people exposed to UV-C radiation, such as industries utilizing germicidal lamps.

The UV-B region (between 290 nm and 320 nm), is generally problematic in that causes sunburn. Extended exposure to UV-B radiation can cause photodamage, photodermatoses and Herpes solaris.

In addition, long-wave UV-A radiation (between 320 nm and 400 nm) has more recently be found to be much more dangerous than UV-B radiation with regard to the triggering of photodynamic, specifically phototoxic reactions and chronic changes in the skin. In fact, because about 90% of the ultraviolet radiation that reaches the Earth consists of UV-A rays, even short exposure during normal everyday conditions can harm the collagen and elastin fibers which are of essential importance for the structure and strength of the skin. The consequences are chronic photo-induced changes in the skin—the skin “ages” prematurely.

Further, while UV-B radiation varies widely depending on numerous factors (e.g. time of year and time of day or degree of latitude), UV-A radiation remains relatively constant day to day irrespective of the time of year and time of day or geographical factors. In addition, the majority of UV-A radiation penetrates into the living epidermis, while approximately 70% of UV-B rays are retained by the horny layer.

When applying a sunscreen to the skin, the ultraviolet rays can be weakened through two effects (1) screening or blocking and (2) absorption. The present disclosure may utilize either or both of these effects, or may solely protect the skin by absorbing UV radiation and converting it to a more beneficial or innocuous radiation, as previously described and discussed in more detail below.

In one embodiment, the disclosed composition can be used as a sunscreen for reducing or preventing the harmful effects of solar radiation on skin. In this embodiment, the composition may comprise an oil-in-water emulsion that includes the previously described ingredients, including the organic and/or inorganic phosphors, instead of or in addition to traditional sunscreen ingredients.

Compositions described herein can also can be used for rejuvenating the skin, independent and together with its use as a sunscreen. In addition, the disclosed compositions can be used with light sources, including fluorescent and incandescent lights, LEDs, diode lasers, such as the 810 nm laser or 1320, 1450, 1720 nm lasers. Depending on the desired treatment, compositions according to the present disclosure may comprise phosphors that change the incident radiation to any wavelength ranging from red to blue. For example, red, orange, yellow, green and blue light has been shown to aid in skin rejuvenation-vein removal, pore reduction, wrinkle reduction, and acne treatment. What has not been shown, until now, is that the incident radiation can be from the sun or from a source that undergoes a change in wavelength primarily because of a topical composition applied to the skin and comprising the disclosed phosphors.

Thus, one embodiment of the present disclosure is directed to a topical composition, such as a sunscreen, comprising disclosed phosphors. To that end, mention is made to known ingredients that can form the base to which the disclosed phosphors are added. For example, a description of conventional topical cream and lotion compositions are provided in Sagarin, Cosmetics Science and Technology, 2nd Edition, Volume 1, Wiley Interscience (1972), and Encyclopedia of Chemical Technology, Third Edition, Volume 7, which is herein incorporated by reference. Descriptions of conventional sunscreen compositions are disclosed in U.S. Pat. Nos. 6,540,986, 6,830,746, and 7,144,570, all of which are herein incorporated by reference.

Non-limiting examples of such materials include one or more of a select group of anionic emulsifiers, such as salts of fatty acids. Mention is made of anionic emulsifiers, including sodium stearate, sodium laurate, sodium lauryl sulfate, DEA cetyl phosphate, dioctyl sulfosuccinate and the like.

The anionic emulsifiers should be present in the compositions of this invention in an amount from about 0.01 to about 10%, such as from 0.5 to about 5%. There may be additional emulsifiers present in the compositions of this invention, such as nonionic emulsifiers known to those of ordinary skill in the art, such as sorbitan esters and ethoxylated sorbitan esters, ethoxylated fatty acids, fatty alcohols and ethoxylated fatty alcohols, fatty glyceride esters and ethoxylated fatty glyceride esters and the like.

In addition, a carrier oil can be present in the compositions of this invention, such fatty acid esters and their derivatives. In one embodiment, the carrier oil is a C₈ to C₂₂ fatty alkyl(optionally polypropylenoxy)polyethylenoxy, ether carboxylate ester, the ester having an alkyl group which has from one to twenty-two carbon atoms, optionally straight or branched. A non-limiting example of this type of carrier oil is isopropyl propylene glycol-2-isodeceth-7 carboxylate, such as Velsan D8P3 or other commercially available materials sold by Sandoz under the Velsan trade name.

In one embodiment, the carrier oil is present in the composition in an amount of ranging from 0.1% to 10%, such as from 1% to 5% by weight of the composition. The oil phase typically contains at least two materials, the polyether carrier oil and a conventional emollient known to those of ordinary skill in the art as useful in sunscreen products, such as mineral oil, ester oils or others known to those of ordinary skill in the art, such as mineral oils, vegetable oils, silicones, synthetic emollients such as fatty acid esters and the like. This emollient should be present in the formulation in a ratio to the carrier concentration of from about 1:1 to about 3:1, such as from about 2:1. The carrier oil and the emollient should compose from about 2% to about 20% of the composition by weight.

In addition to the previously mentioned organic and inorganic phosphors, the inventive sunscreens may also contain traditional oxides known to block and/or scatter harmful radiation. Known UV-blocking agents which may be used in the present invention are described in U.S. Pat. Nos. 6,855,311 and 6,936,241, which are herein incorporated by reference. Non-limiting examples of such UV screening agents include metal oxides chosen from titanium oxide (titanium dioxide in amorphous form or crystallized in rutile and/or anatase form), zinc oxide, iron oxide, zirconium oxide cerium oxide, or mixtures thereof. These well-known metal oxides may be coated or uncoated.

In one embodiment, coatings on the metal oxides may be hydrophobic, e.g., having no affinity for water and which is not made wet by water. This coating may be obtained by one or more surface treatments of the metal oxide with one or more hydrophobic compounds.

The present disclosure is also directed to a method of protecting the skin from the sun, wherein the method comprises a combination of blocking or screening UV radiation and converting UV radiation to radiation having a different wavelength, as previously described. In this embodiment, the inventive composition comprises a combination of known UV blocking agents and the described phosphors.

The present disclosure is also directed to a composition comprising a component for absorbing, such as UV absorbing polymers, in addition to or instead of blocking UV light. In one embodiment, the inventive composition comprises 0.1-15% by weight of a water-soluble, cationic, ultraviolet light absorbing polymers. As described in U.S. Pat. No. 7,008,618, which is herein incorporated by reference, such cationic, ultraviolet light absorbing polymers can be prepared by polymerizing one or more vinyl, allyl or acrylic monomers with one or more vinyl or acrylic monomers that absorb ultraviolet light radiation having a wavelength of about 200 to about 420 nm.

Thus, there is also disclosed a method of protecting the skin from UV radiation that is at least partially based on absorbing the UV radiation. This method may be is in conjunction with or instead of the previously mentioned mechanisms, namely, UV screening.

Additionally, the usual elements of a modem sunscreen emulsion system, such as a polymeric thickener/stabilizer, one or more additional emollient oil, microbial preservatives, antioxidants, fragrance, humectant, waterproofing agents, insect repellants, antimicrobial preservatives, antioxidants, chelating agents, fragrances and moisturizers, suitable carriers for topical application and emulsions.

The compositions of this invention can be in either liquid or aerosol form. They can be incorporated into various cosmetic and personal care products such as hand and body lotions, oils, ointments, lip balm products, facial cosmetics and the like.

In the final sunscreen, product, the organic and inorganic particles will typically have a particle size (on a number basis) less than 1.0 microns, such as an average particle size (on a number basis) ranging from 0.05 to 0.2 microns.

On a mass basis, the finished product should have an average particle size of less than 0.30 microns. In one embodiment, the finished product will have at least 95% of its mass accounted for by particles with diameters of less than 1.0 micron.

In one embodiment, the organic and/or inorganic, non-phosphor particles can be present in the composition in the amount ranging from 0.1% to 25%, such as from 0.5 to 10%. The organic and/or inorganic sunscreen compound should be oil dispersible, and may be present with or without surface coating.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and in the attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result. The percentages are given on a weight basis.

What is claimed is:

1. A topical composition comprising at least one up-converting phosphor in a physiologically acceptable medium.

2. The composition of claim 1, wherein said at least one up-converting phosphor is present in an amount effective to convert electromagnetic radiation of a frequency (A) to a higher frequency (B).

3. The composition of claim 1, wherein said at least one up-converting phosphor is chosen from: sodium yttrium fluoride; lanthanum fluoride; lanthanum oxysulfide (La₂O₂S); yttrium oxysulfide; yttrium fluoride; yttrium gallate; yttrium aluminum garnet (YAG); gadolinium fluoride; barium yttrium fluoride; gadolinium oxysulfide (Gd₂O₂S); calcium tungstate (CaWO₄); yttrium oxide:terbium (Yt₂O₃Tb); gadolinium oxysulphide: europium (Gd₂O₂S:Eu); lanthanam oxysulphide: europium (La₂O₂S:Eu); and gadolinium oxysulphide: promethium, cerium, fluorine; Zn₂SiO₄:Mn; CaWO₄; (Zn, Cd)S:Cu; ZnS:Ag; ZnO:Zn; KMgF₃:Mn; (Zn, Cd)S:Ag; (Zn, Cd)S:Ag; ZnS:Ag; ZnS:Cu,Au,Al; YVO₄:Eu; Y₂O₂S:Eu; CaSiO₃:Mn,Pb; KMgF₃:Mn; ZnS:Cu; MgF₂:Mn; (Zn,Mg)F₂:Mn; Zn₂SiO₄:Mn,As; Zn₂SiO₄:Mn,In; Gd₂O₂S:Tb; La₂O₂S:Tb; Y₂O₂S:Tb; Y₂O₂S:Tb,Eu; Y₃Al₅O₁₂:Ce; Y₃(Al,Ga)₅O₁₂:Ce; Y₂SiO₅:Ce; Y₃(Al,Ga)₅O₁₂:Tb; ZnS:Ag; Y₂O₃:Eu; InBO₃:Eu; InBO₃:Tb; InBO₃:Eu+InBO₃:Tb+ZnS:Ag; InBO₃:Eu+InBO₃:Tb; Y₂O₂S:Yb; Y₂O₂S:Yb,Er; Y₂O₂S:Yb,Ho; La₂O₂S:Yb,Er; La₂O₂S:Yb; Gd₂O₂S:Yb,Ho; Gd₂O₂S:Yb,Er; Gd₂O₂S:Yb; Gd₂O₂S:Tb; Gd₂O₂S:Eu; Gd₂O₂S:Pr; La₂O₂S:Eu; La₂O₂S:Tb; Y₂O₂S:Tb; (Zn, Cd)S:Ag; CsI:Na; CsI:Tl; CaS:Eu,Sm; CaS:Ce,Sm; SrS:Eu,Sm; ZnS:Cu,Pb; and ZnS:Cu,Pb,Mn.

4. The composition of claim 1, wherein said at least one up-converting phosphor comprises a ytterbium containing activator.

5. The composition of claim 4, wherein said ytterbium containing activator is chosen from ytterbium/erbium, ytterbium/thulium, ytterbium/terbium, and ytterbium/holmium.

6. The composition of claim 1, wherein the electromagnetic radiation of frequency (A) comprises infrared radiation or visible light.

7. The composition of claim 1, wherein the electromagnetic radiation of frequency (A) comprises ionizing radiation chosen from gamma radiation and x-rays.

8. The composition of claim 1, wherein the electromagnetic radiation of frequency (B) comprises ultraviolet (UV) radiation chosen from UVA and UVB.

9. The composition of claim 1, wherein the electromagnetic radiation of frequency (A) comprises UVC.

10. The composition of claim 1, further comprising one or more psoralen compounds or psoralen derivatives.

11. The composition of claim 10, wherein said one or more psoralen compounds or psoralen derivatives are chosen from 8-methoxypsoralen (8-MOP), 5-methoxypsoralen (5-MOP), and tri-psoralen.

12. The composition of claim 1, wherein said at least one up-converting phosphor has an average particle size ranging from 5 to 1000 nanometers.

13. The composition of claim 1, wherein said at least one up-converting phosphor is present in an amount ranging from 0.01% to 60% by weight, relative to the total weight of the composition.

14. The composition of claim 13, wherein said at least one up-converting phosphor is present in an amount ranging from 0.1% to 30% by weight, relative to the total weight of the composition.

15. The composition of claim 13, wherein said at least one up-converting phosphor is present in an amount ranging from 1% to 15% by weight, relative to the total weight of the composition.

16. The composition of claim 1, further comprising at least one film-forming polymer.

17. The composition according to claim 16, wherein said at least one film-forming polymer is dissolved or dispersed in said physiologically acceptable medium.

18. The composition of claim 16, wherein the at least one film-forming polymer is chosen from vinyl polymers, acrylic polymers, polyurethanes, polyureas, polyesters, polyesteramides, polyamides, epoxyester resins, and cellulose polymers.

19. The composition of claim 16, wherein said at least one film-forming polymer is present in an amount ranging from 1% to 50% by weight, relative to the total weight of the composition.

20. The composition of claim 1, wherein said physiologically acceptable medium is chosen from at least one solvent, water and mixtures thereof.

21. The composition of claim 20, wherein said at least one solvent is chosen from organic solvents.

22. The composition of claim 1, wherein the physiologically acceptable medium is chosen from aqueous-alcoholic mixtures.

23. The composition of claim 1, wherein said physiologically acceptable medium is present in an amount ranging from 25% to 99% by weight, relative to the total weight of the composition.

24. The composition of claim 1, further comprising at least one plasticizer and/or coalescers in an amount ranging from 0.5% to 20% by weight, relative to the total weight of the composition.

25. The composition of claim 1, further comprising at least one additive chosen from vitamins, minerals, thickeners, fillers, spreading agents, thixotropes, rheological agents, wetting agents, dispersants, anti-foaming agents, preserving agents, UV-screening agents, UV-absorbing agents, active agents, surfactants, moisturizers, fragrances, neutralizing agents, stabilizers, and antioxidants.

26. The composition of claim 1, wherein said composition is a skin care product, a hair care product, sun care product, oral care product, a personal cleaning product, or a sunscreen.

27. A topical composition comprising at least one down-converting phosphor in a physiologically acceptable medium.

28. The composition of claim 27, wherein said at least one down-converting phosphor is chosen from: sodium yttrium fluoride; lanthanum fluoride; lanthanum oxysulfide (La₂O₂S); yttrium oxysulfide; yttrium fluoride; yttrium gallate; yttrium aluminum garnet (YAG); gadolinium fluoride; barium yttrium fluoride; gadolinium oxysulfide (Gd₂O₂S); calcium tungstate (CaWO₄); yttrium oxide:terbium (Yt₂O₃Tb); gadolinium oxysulphide: europium (Gd₂O₂S:Eu); lanthanam oxysulphide: europium (La₂O₂S:Eu); and gadolinium oxysulphide: promethium, cerium, fluorine; Zn₂SiO₄:Mn; CaWO₄; (Zn, Cd)S:Cu; ZnS:Ag; ZnO:Zn; KMgF₃:Mn; (Zn, Cd)S:Ag; (Zn, Cd)S:Ag; ZnS:Ag; ZnS:Cu,Au,Al; YVO₄:Eu; Y₂O₂S:Eu; CaSiO₃:Mn,Pb; KMgF₃:Mn; ZnS:Cu; MgF₂:Mn; (Zn,Mg)F₂:Mn; Zn₂SiO₄:Mn,As; Zn₂SiO₄:Mn,In; Gd₂O₂S:Tb; La₂O₂S:Tb; Y₂O₂S:Tb; Y₂O₂S:Tb,Eu; Y₃Al₅O₁₂:Ce; Y₃(Al,Ga)₅O₁₂:Ce; Y₂SiO₅:Ce; Y₃(Al,Ga)₅O₁₂:Tb; ZnS:Ag; Y₂O₃:Eu; InBO₃:Eu; InBO₃:Tb; InBO₃:Eu+InBO₃:Tb+ZnS:Ag; InBO₃:Eu+InBO₃:Tb; Y₂O₂S:Yb; Y₂O₂S:Yb,Er; Y₂O₂S:Yb,Ho; La₂O₂S:Yb,Er; La₂O₂S:Yb; Gd₂O₂S:Yb,Ho; Gd₂O₂S:Yb,Er; Gd₂O₂S:Yb; Gd₂O₂S:Tb; Gd₂O₂S:Eu; Gd₂O₂S:Pr; La₂O₂S:Eu; La₂O₂S:Tb; Y₂O₂S:Tb; (Zn, Cd)S:Ag; CsI:Na; CsI:Tl; CaS:Eu,Sm; CaS:Ce,Sm; SrS:Eu,Sm; ZnS:Cu,Pb; and ZnS:Cu,Pb,Mn.

29. The composition of claim 27, wherein said at least one down-converting phosphor comprises a ytterbium containing activator.

30. The composition of claim 27, further comprising one or more psoralen compounds or psoralen derivatives.

31. The composition of claim 30, wherein said one or more psoralen compounds or psoralen derivatives are chosen from 8-methoxypsoralen (8-MOP), 5-methoxypsoralen (5-MOP), and tri-psoralen.

32. The composition of claim 28, wherein said at least one down-converting phosphor has an average particle size ranging from 5 to 1000 nanometers.

33. The composition of claim 27, wherein said at least one down-converting phosphor is present in an amount ranging from 0.01% to 60% by weight, relative to the total weight of the composition.

34. The composition of claim 27, further comprising at least one film-forming polymer.

35. The composition of claim 27, wherein said physiologically acceptable medium is chosen from at least one solvent, water and mixtures thereof.

36. The composition of claim 27, further comprising at least one additive chosen from vitamins, minerals, thickeners, fillers, spreading agents, thixotropes, rheological agents, wetting agents, dispersants, anti-foaming agents, preserving agents, UV-screening agents, UV-absorbing agents, active agents, surfactants, moisturizers, fragrances, neutralizing agents, stabilizers, and antioxidants.

37. The composition of claim 27, wherein said composition is a skin care product, a hair care product, sun care product, oral care product, a personal cleaning product, or a sunscreen.

38. A method for electromagnetically treating keratinous material, said method comprising:
applying to at least one keratinous surface, a topical composition comprising at least one organic and/or inorganic phosphor in a physiologically acceptable medium, wherein said at least one phosphor is present in an amount effective to convert electromagnetic radiation of an initial frequency to a different frequency; and
exposing said keratinous material to a source of electromagnetic radiation with said initial frequency.

39. The method of claim 38, wherein said at least one organic and/or inorganic phosphor is chosen from: sodium yttrium fluoride; lanthanum fluoride; lanthanum oxysulfide (La₂O₂S); yttrium oxysulfide; yttrium fluoride; yttrium gallate; yttrium aluminum garnet (YAG); gadolinium fluoride; barium yttrium fluoride; gadolinium oxysulfide (Gd₂O₂S); calcium tungstate (CaWO₄); yttrium oxide:terbium (Yt₂O₃Tb); gadolinium oxysulphide: europium (Gd₂O₂S:Eu); lanthanam oxysulphide: europium (La₂O₂S:Eu); and gadolinium oxysulphide: promethium, cerium, fluorine; Zn₂SiO₄:Mn; CaWO₄; (Zn,Cd)S:Cu; ZnS:Ag; ZnO:Zn; KMgF₃:Mn; (Zn, Cd)S:Ag; (Zn, Cd)S:Ag; ZnS:Ag; ZnS:Cu,Au,Al; YVO₄:Eu; Y₂O₂S:Eu; CaSiO₃:Mn,Pb; KMgF₃:Mn; ZnS:Cu; MgF₂:Mn; (Zn,Mg)F₂:Mn; Zn₂SiO₄:Mn,As; Zn₂SiO₄:Mn,In; Gd₂O₂S:Tb; La₂O₂S:Tb; Y₂O₂S:Tb; Y₂O₂S:Tb,Eu; Y₃Al₅O₁₂:Ce; Y₃(Al,Ga)₅O₁₂:Ce; Y₂SiO₅:Ce; Y₃(Al,Ga)₅O₁₂:Tb; ZnS:Ag; Y₂O₃:Eu; InBO₃:Eu; InBO₃:Tb; InBO₃:Eu+InBO₃:Tb+ZnS:Ag; InBO₃:Eu+InBO₃:Tb; Y₂O₂S:Yb; Y₂O₂S:Yb,Er; Y₂O₂S:Yb,Ho; La₂O₂S:Yb,Er; La₂O₂S:Yb; Gd₂O₂S:Yb,Ho; Gd₂O₂S:Yb,Er; Gd₂O₂S:Yb; Gd₂O₂S:Tb; Gd₂O₂S:Eu; Gd₂O₂S:Pr; La₂O₂S:Eu; La₂O₂S:Tb; Y₂O₂S:Tb; (Zn, Cd)S:Ag; CsI:Na; CsI:Tl; CaS:Eu,Sm; CaS:Ce,Sm; SrS:Eu,Sm; ZnS:Cu,Pb; and ZnS:Cu,Pb,Mn.

40. The method of claim 38, wherein said at least one organic and/or inorganic phosphor comprise a ytterbium containing activator.

41. The method of claim 40, wherein said ytterbium containing activator is chosen from ytterbium/erbium, ytterbium/thulium, ytterbium/terbium, and ytterbium/holmium.

42. The method of claim 38, wherein said at least one organic and/or inorganic phosphor is up-converting and the electromagnetic radiation of said initial frequency comprises infrared or visible light and said different frequency comprises ultraviolet (UV) radiation.

43. The method of claim 38, wherein said at least one organic and/or inorganic phosphor is down-converting and the electromagnetic radiation of said initial frequency comprises ultraviolet (UV) radiation and said different frequency ranges from visible light to IR radiation.

44. The method of claim 38, wherein said topical composition further comprises one or more psoralen compounds or psoralen derivatives.

45. The method of claim 44, wherein said one or more psoralen compounds or psoralen derivatives are chosen from 8-methoxypsoralen (8-MOP), 5-methoxypsoralen (5-MOP), and tri-psoralen.

46. The method of claim 38, wherein said at least one organic and/or inorganic phosphor has an average particle size ranging from 5 to 1000 nanometers.

47. The method of claim 38, wherein said at least one organic and/or inorganic phosphor is present in an amount ranging from 0.01% to 60% by weight, relative to the total weight of the composition.

48. The method of claim 47, wherein said at least one organic and/or inorganic phosphor is present in an amount ranging from 0.1% to 30% by weight, relative to the total weight of the composition.

49. The method of claim 48, wherein said at least one organic and/or inorganic phosphor is present in an amount ranging from 1% to 15% by weight, relative to the total weight of the composition.

50. The method of claim 38, wherein said topical composition further comprises at least one film-forming polymer.

51. The method of claim 38, wherein said physiologically acceptable medium is chosen from at least one solvent, water and mixtures thereof.

52. The method of claim 51, wherein said at least one solvent is chosen from organic solvents.

53. The method of claim 38, wherein the physiologically acceptable medium is chosen from aqueous-alcoholic mixtures.

54. The method of claim 53, wherein said aqueous-alcoholic mixture comprises at least one C₁-C₅ monoalcohol.

55. The method of claim 38, wherein said topical composition further comprises at least one plasticizer and/or coalescers in an amount ranging from 0.5% to 20% by weight, relative to the total weight of the composition.

56. The method of claim 38, wherein said topical composition further comprises at least one additive chosen from vitamins, minerals, thickeners, fillers, spreading agents, thixotropes, rheological agents, wetting agents, dispersants, anti-foaming agents, preserving agents, UV-screening agents, UV-absorbing agents, active agents, surfactants, moisturizers, fragrances, neutralizing agents, stabilizers, and antioxidants.

57. The method of claim 38, wherein said topical composition is in the form of a powder, liquid, solution, cream, gel, ointment, serum, or transdermal patch.

58. A method for protecting keratinous material from UV radiation, said method comprising:
applying to at least said keratinous material, a topical composition comprising, in a physiologically acceptable medium:
a) at least one organic and/or inorganic phosphor that converts electromagnetic radiation of an initial frequency to a different frequency; and
b) a UV-absorbing material, a UV-blocking material, or combinations thereof.

59. A method for treating at least one skin condition, said method comprising:
applying to at least one keratinous surface, a topical composition comprising, in a physiologically acceptable medium, at least one organic and/or inorganic phosphor that converts electromagnetic radiation of an initial frequency to a different frequency, said method comprising exposing the skin to a source of electromagnetic radiation with said initial frequency.
